(19) United States
(12) Patent Application Publication
(10) Pub. No.: US 2006/0008432 A1
(43) Pub. Date: Jan. 12, 2006

(54) Gilsonite derived pharmaceutical delivery compositions and methods: nail applications

(76) Inventors: Sebastiano Scarampi, San Francisco, CA (US); Peter M. Elias, San Francisco, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/887,466

(22) Filed: Jul. 7, 2004

(51) Int. Cl. A61K 8/31

(52) U.S. Cl. 424/61

(57) ABSTRACT

Pharmaceutical formulations comprising Gilsonite oil for topical or transdermal application are disclosed. Also provided are methods for transdermal administration of a bioactive agent with enhanced penetration which comprise topical administration of a composition comprising transdermally effective amount of Gilsonite oil.

This application is related to U.S. application Ser. No. 10/751,276, filed Jan. 2, 2004, which is hereby incorporated-by-reference for all purposes.

### BACKGROUND OF THE INVENTION

1. Field of the Invention

The present invention relates to pharmaceutical compositions for transdermal, transmucosal and topical delivery, and methods of use of the same. More specifically, these compositions comprise a penetration enhancing agent and a bioactive agent.

2. State of the Art

The transdermal route of parenteral delivery of drugs provides many advantages, and examples of transdermal systems for delivering a wide variety of drugs are described in U.S. Pat. Nos. 3,598,122; 3,598,123; 3,731,683; 3,797,494; 4,286,592; 4,314,557; 4,379,454; 4,435,180; 4,559,222; 4,568,343; 4,573,999; 4,588,580; 4,645,502; 4,704,282; 4,816,258; 4,849,226; 4,908,027; 4,943,435; 5,004,610; 5,006,342; 5,314,694; 5,411,740, 5,629,019; 5,641,504; 5,686,097, the disclosures of which are incorporated herein by reference. In many cases, drugs which would appear to be ideal candidates for transdermal delivery are found to have such low permeability through intact skin that they cannot be delivered in therapeutically effective amounts from reasonably sized devices or applications of material in reasonable amounts.

In an effort to increase skin permeability so that drugs can be delivered transdermally in therapeutically effective amounts, it has been proposed to pretreat the skin with various chemicals or to concurrently deliver the drug in the presence of a permeation enhancer. Various materials have been suggested for this use, as described in U.S. Pat. Nos. 3,472,931; 3,527,864; 3,896,238; 3,903,256; 3,952,099; 4,046,886; 4,130,643; 4,130,667; 4,299,826; 4,335,115; 4,343,798; 4,379,454; 4,405,616; 4,568,343; 4,746,515; 4,764,379; 4,788,062; 4,820,720; 4,863,738; 4,863,970; 4,865,848; 4,900,555; 4,940,586; 4,973,468; 5,053,227; 5,059,426; 5,378,730; and WO 95/01167, the disclosures of which are hereby incorporated in their entirety by reference. Williams et al. “Skin Absorption Enhancers” Critical Review in Therapeutic Drug Carrier Systems, pp. 305-353 (1992) and Santus et al. “Transdermal Enhancer Patent Literature”, Journal of Controlled Release, pp. 1-20 (1993) also provide a review of transdermal permeation enhancers.

To be considered useful, a permeation enhancer should have the ability to enhance the permeability of the skin for at least one and preferably a significant number of drugs. More importantly, it should be able to enhance the skin permeability such that the drug delivery rate from a reasonably sized system (preferably 5-60 cm²) is at therapeutically effective levels. Additionally, the ideal permeation enhancer when applied to the skin surface, should be non-toxic, non-irritating on prolonged exposure and under occlusion, and non-sensitizing on repeated exposure. Preferably, it should be odorless, physiologically inactive, and capable of delivering drugs without producing sensations such as, burning or tingling, or other irritating sensations. DMSO has been promoted as an effective permeation enhancer or carrier for transdermal drug delivery, but it is undesirable due to toxicity concerns.

In addition to these permeation enhancer-skin interaction considerations, a permeation enhancer should also be evaluated with respect to possible interactions within the transdermal system itself, such as a patch adhered to the skin by an adhesive. For example, the permeation enhancer should be compatible with the drug to be delivered, the patch adhesive, and the polymer matrix or other reservoir from which the drug is dispensed to the skin. The permeation enhancer should also be selected so as to contribute to, or at best not unduly interfere with, the suitable balance among tack, adhesion, and cohesive strength of the adhesive.

The use of a cosolvent in combination with a permeation enhancer has also been disclosed. Such cosolvents may not appreciably increase transdermal flux by themselves, but may act synergistically to increase the transdermal flux of a drug when used in combination with other permeation enhancers. One theory is that these cosolvents act to increase the availability of the permeation enhancer at the skin surface, thus providing increased flux of drug.

For example, WO 95/09006 discloses the use of various lactic acid ester cosolvents such as lauryl lactate, ethyl lactate, cetyl lactate, and myristyl lactate in combination with a monoglyceride. However, these lactic acid esters may be irritating to the skin. WO 96/40259 discloses the use of lauryl acetate as a cosolvent for monoglyceride permeation enhancers such as glycerol monolaurate. This combination provides enhanced flux when compared to other monoglyceride/cosolvent combinations and is available at a high degree of purity. But lauryl acetate has been found to be an undesirable cosolvent from a manufacturing standpoint. It has been found that an undesirable amount of lauryl acetate evaporates during manufacturing of transdermal delivery systems due to its high vapor pressure, leaving insufficient amounts of lauryl acetate in the system.

Therefore, in spite of these advances, problems associated with skin irritation and more recently discovered problems associated with processing and manufacturing of films comprising various cosolvents for monoglycerides have left a need for alternative penetration enhancing formulations.

Additionally, U.S. Pat. No. 5,312,122 discloses the use of monoglycerides and fatty acid esters, alone or in combination, as a permeation enhancer mixture for a synthetic progestogen. U.S. Pat. No. 5,026,556 discloses a composition for the transdermal delivery of buprenorphine comprising an amount of buprenorphine in a carrier comprising a polar solvent material selected from the group consisting of C₃-C₄ diols, C₃-C₆ triols, and mixtures thereof; and a polar lipid material selected from the group consisting of fatty alcohol esters, fatty acid esters, and mixtures thereof. Ethyl palmitate is disclosed as a suitable polar lipid material.

U.S. Pat. No. 5,352,456 discloses a transdermal device which provides an initial pulse of drug followed by a substantially lower continuous rate. The device comprises a drug reservoir comprising the drug dissolved in a carrier and a volatile permeation enhancer. The volatile permeation enhancer is depleted from the reservoir by evaporation through the backing layer causing the decrease in drug delivery rate. The volatile permeation enhancers are described as having a vapor pressure of greater than about 10 mm Hg at 25° C.

U.S. Pat. No. 5,149,538 discloses the transdermal delivery of an opioid with preferred permeation enhancers comprising saturated and unsaturated fatty alcohols, fatty alcohol esters, or fatty acids having 8-18 carbon atoms. U.S. Pat. No. 5,650,165 discloses percutaneous absorption preparations comprising an acrylic copolymer, a fatty acid ester comprising a higher fatty acid having 12-16 carbon atoms and a lower monohydric alcohol having 1-4 carbon atoms, and a monoglyceride comprising a higher fatty acid having 8-10 carbon atoms. U.S. Pat. No. 5,747,069 discloses a percutaneous absorbable preparation containing a drug and a permeation enhancer, comprising a monoglyceride and a fatty acid. The disclosures of the above referenced patents are incorporated herein in their entirety by reference.

In view of the above, it is apparent that there is a need for new and improved systems for transdermal drug delivery.

### SUMMARY OF THE INVENTION

The present invention provides pharmaceutical formulations comprising a Gilsonite oil and a bioactive agent for topical application for transdermal delivery of the bioactive agent. Also provided are methods for transdermal administration of a bioactive agent with enhanced penetration which comprise topical administration of a composition comprising transdermally effective amount of a Gilsonite oil for transdermal delivery of the bioactive agent. As used herein “a Gilsonite oil” means a type of oil derived from uintaite materials and available from the American Gilsonite Company, San Francisco, Calif. Gilsonite oils are available in various grades having different physical properties, such as viscosity. Following the teachings of this disclosure the appropriate Gilsonite oil or oils can be selected for formulation with the desired bioactive agent and other components to provide an effective transdermal delivery system according to this invention. Gilsonite oils of different properties can be used in mixtures in this invention, but the disclosure herein is directed to using a single Gilsonite oil in each formulation in order to teach and illustrate the present invention.

In one of the composition aspects, the present invention provides a composition for topical or transdermal administration comprising a bioactive agent and a Gilsonite oil. Preferably the composition further comprises a pharmaceutically acceptable carrier, such as a conventional oil, gel, cream, ointment, lotion, adhesive, polymer, paste, or spray that may optionally contain one or more pharmaceutically acceptable additives selected from the group consisting of excipients, preservatives, antioxidants, fragrances, emulsifiers, dyes, anti-irritants and additional penetration enhancers. The bioactive agent component is present in an effective amount for the intended use, preferably at least about 0.01% by weight, preferably about 0.1% to about 50%, and the Gilsonite oil is preferably present in at least about 0.01% by weight, preferably about 0.1% to about 50%. In some formulations the Gilsonite oil component can be present as the major proportion ingredient (possibly even the only ingredient besides the bioactive agent) and thus serve the functions of both the penetration enhancer and carrier or diluent.

In another of the composition aspects, the present invention provides a penetration-enhancing system comprising a Gilsonite oil and a pharmaceutically acceptable carrier, such as a conventional oil, gel, cream, ointment, lotion, adhesive, polymer, paste, or spray that may optionally contain one or more pharmaceutically acceptable additives selected from the group consisting of excipients, preservatives, antioxidants, fragrances, emulsifiers, dyes, anti-irritants, and additional penetration enhancers. The carrier component is present in at least about 0.1% by weight, preferably about 1% to about 80%, more preferable about 2% to about 50%, and the Gilsonite oil component is present in at least about 0.1% by weight, preferably about 1% to about 80%, and more preferably about 2% to about 50%. This composition of Gilsonite oil and pharmaceutically acceptable carrier provides a base material according to the present invention which can be formulated with a desired bioactive agent.

In still another of the composition aspects, the present invention provides a composition for topical or transdermal administration with enhanced penetration comprising Gilsonite oil and a bioactive agent formulated with a penetration enhancing system comprising a Gilsonite oil and a pharmaceutically acceptable carrier, such as a conventional oil, gel, cream, ointment, lotion, adhesive, polymer, paste, or spray that may optionally contain one or more pharmaceutically acceptable additives selected from the group consisting of excipients, preservatives, antioxidants, fragrances, emulsifiers, dyes, anti-irritants, and additional penetration enhancers. The Gilsonite oil-bioactive agent component and the Gilsonite oil-carrier component can each be formulated as described above, then these two components can be mixed or formulated in a weight ratio between about 1:100 and about 100:1, preferably about 10:90 to about 90:10 and more preferably about 20:80 to about 80:20.

In one of the method aspects, the present invention provides a method for enhancing the penetration of a bioactive agent through the skin or nail of a human or other animal which comprises applying to the skin or nail of the animal a composition comprising a bioactive agent and a penetration-enhancing system containing Gilsonite oil, as described herein. Similarly, the present invention provides a method for enhancing the penetration of a bioactive agent into a plant system which comprises applying to the surface or a cut opening of a plant member a composition comprising a bioactive agent and a penetration-enhancing system containing Gilsonite oil, as described herein.

In another method aspect, the present invention provides a method for delivering a bioactive agent transdermally by administering to the skin or nail of an animal a composition comprising a bioactive agent and Gilsonite oil, as described herein, and provides a method of delivering a bioactive agent into a plant system by applying to the surface or a cut opening of a plant member a composition comprising a bioactive agent and Gilsonite oil, as described herein. The methods of delivery according to this aspect of the invention include direct application of the composition, application from an applicator device, time release application from an applicator device and other methods adapted according to the disclosure herein.

In another method aspect, the present invention provides a method of making a composition for delivery of a bioactive agent comprising mixing the bioactive agent and Gilsonite oil in proportions effective to provide a composition for the desired use, as described herein. Optionally, additional penetration-enhancing additives, or other pharmaceutically acceptable additives, such as referred to above, may also be mixed with the components or the composition, as described herein.

In another aspect, the present invention provides a device comprising an applicator device, such as a patch, strip or container containing a composition comprising a Gilsonite oil and a bioactive agent and adapted for placement on the skin or nail of the animal or on a plant member and for delivering the composition.

Another aspect of the invention provides a method of making a device for administration of a bioactive agent comprising providing an applicator device adapted for placement on the skin or nail of an animal or on a plant member and incorporating into the applicator a composition comprising Gilsonite oil and a bioactive agent, as disclosed herein.

### BRIEF DESCRIPTION OF THE DRAWINGS

FIG. 1A: A typical Transepidermal Water Loss or “TEWL” vs. time graph.

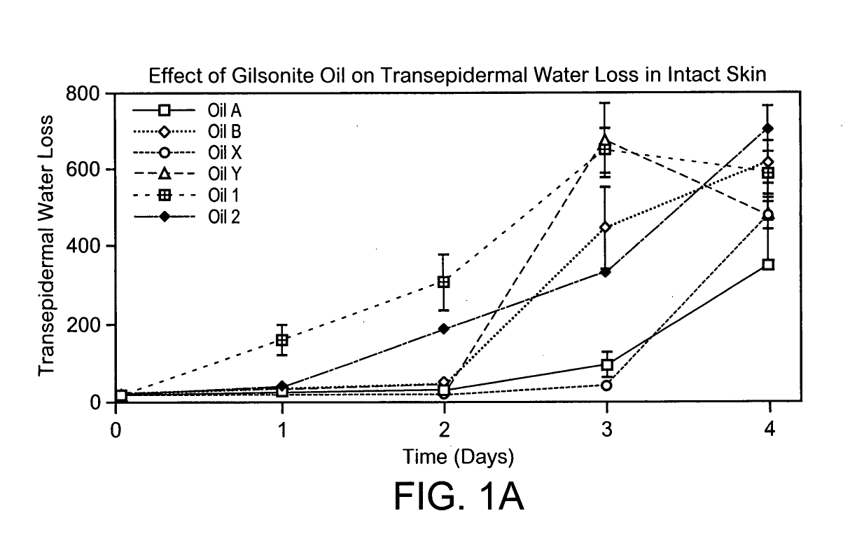

FIG. 1B: A graph showing the effects of Gilsonite oils on skin hydration.

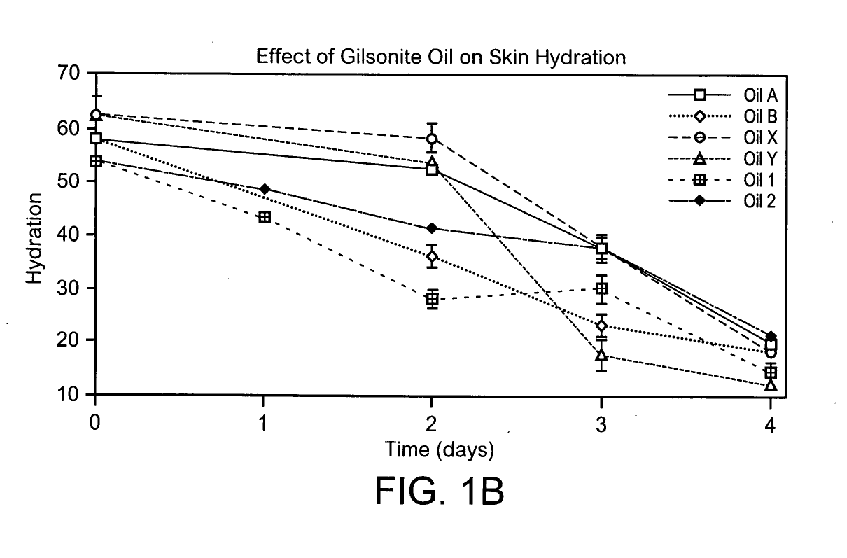

FIG. 2: A graph showing the percent penetration over 12 hours of polyethylene glycol (PEG) molecules as a function of molecular weight.

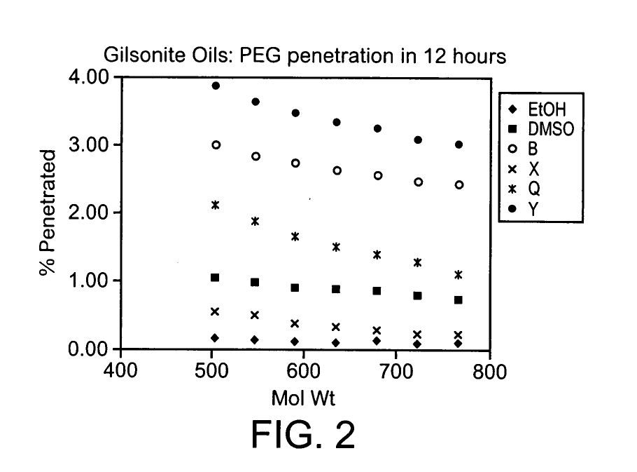

FIG. 3: A graph showing the total hydrocortisone permeation from a Gilsonite oil composition comprising hydrocortisone over 12 h in a porcine skin sample.

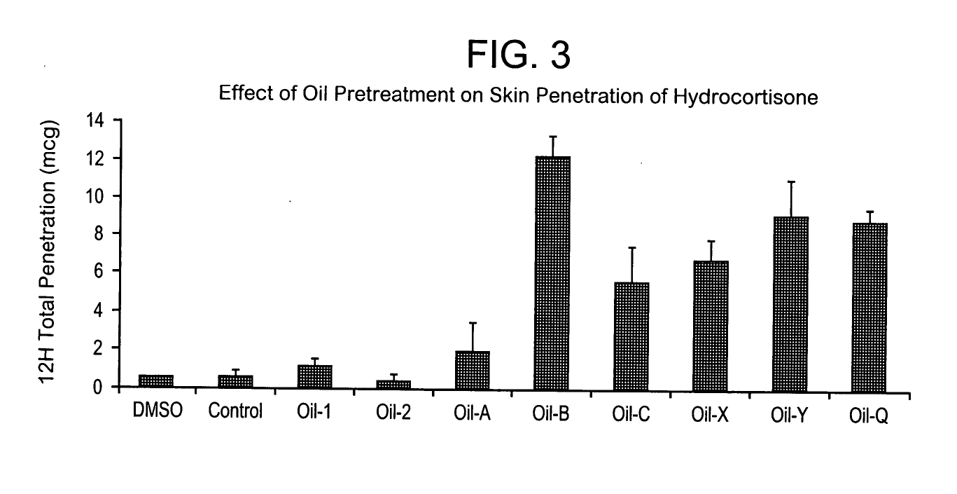

FIG. 4: A graph showing the total caffeine permeation from a Gilsonite oil composition comprising caffeine over 12 h in a porcine skin sample.

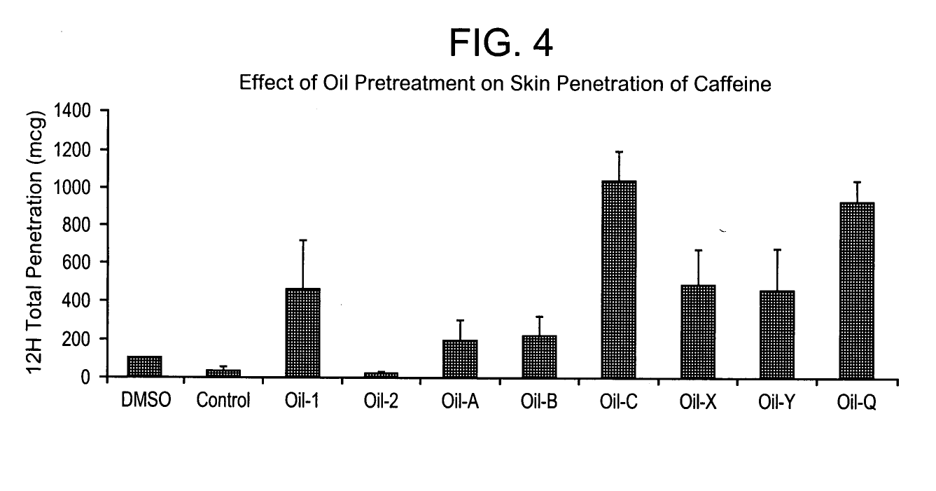

### DESCRIPTION OF THE INVENTION

According to this invention, various Gilsonite oils can be used to effectively provide transdermal delivery of drugs and/or enhance the permeability of drugs through body surfaces, particularly through the skin and through fingernails and toenails. First, the following terms as used herein shall have the following meanings.

The term “topical administration” refers to the application of a pharmaceutical agent to the external surface of the skin or the mucous membranes (including the surface membranes of the nose, lungs and mouth), such that the agent crosses the external surface of the skin or mucous membrane and enters the underlying tissues. Topical administration includes application of the composition to intact skin or mucous membrane, to broken, raw or open wound of skin or mucous membrane. Topical administration of a pharmaceutical agent can result in a limited distribution of the agent to the skin and surrounding tissues or, when the agent is removed from the treatment area by the bloodstream, can result in systemic distribution of the agent. In a preferred form of topical administration, the pharmaceutical agent is delivered by transdermal delivery.

The term “transdermal delivery” refers to the diffusion of an agent across the barrier of the skin resulting from topical administration or other application of a composition. The stratum corneum acts as a barrier and few pharmaceutical agents are able to penetrate intact skin. In contrast, the epidermis and dermis are permeable to many solutes and absorption of drugs therefore occurs more readily through skin that is abraded or otherwise stripped of the stratum corneum to expose the epidermis. Transdermal delivery includes injection or other delivery through any portion of the skin or mucous membrane and absorption or permeation through the remaining portion. Absorption through intact skin can be enhanced by placing the active agent in an appropriate pharmaceutically acceptable vehicle before application to the skin. Passive topical administration may consist of applying the active agent directly to the treatment site in combination with emollients or penetration enhancers. As used herein, reference to transdermal delivery is intended to include delivery by permeation through fingernail and toenail surfaces.

The term “transmucosal delivery” refers to diffusion of an agent across the barrier of the mucosa. The epithelia acts as a barrier and many pharmaceutical agents are unable to penetrate intact mucosa. Absorption through intact mucosa can be enhanced by placing the active ingredient in a vehicle before application to the mucosa. Passive topical administration may consist of applying the active agent directly to the treatment site in combination with penetration enhancers.

The terms “enhancement,” “penetration enhancement” or “permeation enhancement” relate to an increase in the permeability of the skin to a drug, so as to increase the rate at which the drug permeates through the skin. The enhanced permeation effected through the use of such enhancers can be observed, for example, by measuring the rate of diffusion of the drug through animal or human skin using a diffusion cell apparatus. A diffusion cell is described by Merritt et al. Diffusion Apparatus for Skin Penetration, J. of Controlled Release, 1 (1984) pp. 161-162.

The term “permeation enhancer” or “penetration enhancer” intends an agent or a mixture of agents which, alone or in combination, acts to increase the permeability of the skin to a drug.

The terms “drug,” “bioactive agent” or “pharmaceutical agent” refer to any chemical material, compound or composition suitable for transdermal administration which provides a desired biological, pharmacological or nutritional effect. The terms “drug,” “bioactive agent” or “pharmaceutical agent” are also meant to include mixtures of more than one bioactive agent.

The term “matrix”, refers to a composition that is homogeneously or gradiently combined in a biocompatible pressure sensitive adhesive in which the enhancer is homogeneously dissolved or suspended and which may or may not contain other ingredients. A matrix system is usually an occlusive adhesive patch having an impermeable film backing and, before transdermal application, a release liner on the surface of the adhesive opposite the film backing. A matrix system therefore is a unit dosage form of a drug composition in an adhesive carrier, also containing the enhancer and other components which are formulated for maintaining the drug composition in the adhesive in a drug transferring relationship with the derma, skin or nail. Adhesive patches having non-occlusive backings are also considered to be within the scope of this definition unless specifically excluded. The terms “reservoir,” “reservoir patch” or “reservoir system” refer to a pharmaceutical composition in a fluid of controlled viscosity contained in an occlusive device having an impermeable back surface and an opposite surface configured appropriately with permeable membranes and adhesives for transdermal application. A reservoir system therefore is a unit dosage form of a drug composition in a fluid carrier of controlled viscosity, also containing the enhancer and other components which is formulated in an occlusive device for maintaining the drug composition in the carrier in a drug transferring relationship with the derma or skin.

The term “fluid of controlled viscosity” refers to a carrier in which the pharmaceutical formulation is contained in a single or phase separated fluid state. The fluid, per se may serve as a solvent, or a solvent or co-solvent may be added. Such fluids can be water or organic based and may contain a mixture of liquids or solvents appropriately gelled or thickened. In other words, such fluids may comprise, but are not limited to, solutions, suspensions, emulsions, gels, ointments, creams, pastes or any other similar state which permits the outward diffusion of the permeant and enhancer and, optionally, a solvent or other additives as desired.

By “effective” amount of a drug or permeant is meant a sufficient amount of a bioactive agent to provide the desired local or systemic effect. An “effective” amount of permeation enhancer as used herein means an amount selected so as to provide the desired increase in transdermal permeability and, correspondingly, the desired depth of penetration, rate of administration and amount of drug. A “therapeutically effective” amount or rate refers to the amount or rate of drug needed to effect the desired therapeutic result.

The terms “drug delivery system,” “drug enhancer composition” or any similar terminology refer to a formulated composition containing the drug to be transdermally delivered in combination with such “pharmaceutically acceptable carriers” or “pharmaceutically acceptable vehicles”, penetration enhancers, excipients, or any other additives.

The term “pharmaceutically acceptable carrier or vehicle” refers to any formulation or carrier medium that provides the appropriate delivery of an effective amount of a bioactive agent as defined herein, does not interfere with the effectiveness of the biological activity of the bioactive agent, and that is sufficiently non-toxic to the host or patient. One skilled in the art may formulate the compositions of the present invention in an appropriate manner, and in accordance with accepted practices, such as those disclosed in Remington: The Science and Practice of Pharmacy, Gennaro, ed., Mack Publishing Co., Easton, Pa., 19th ed., 1995.

The term “pharmaceutically acceptable additive” refers to preservatives, antioxidants, fragrances, emulsifiers, dyes and excipients known or used in the field of drug formulation and that do not unduly interfere with the effectiveness of the biological activity of the bioactive agent, and that is sufficiently non-toxic to the host or patient.

The term “excipients” is conventionally known to mean carriers, diluents and/or vehicles used in formulating drug compositions effective for the desired use.

Preparation and Formulation

The particular Gilsonite oils referred to herein and used in the Examples set forth herein to illustrate the practice of this invention are designated as Gilsonite oils 1, 2, A, B, C, X, Y, and Q and are materials available from American Gilsonite Corporation, San Francisco, Calif. Each oil has unique physical characteristics such as color and viscosity. Gilsonite oil is manufactured by American Gilsonite Corporation in proprietary processes which produce the various individual Gilsonite oil products having differing physical characteristics. The available Gilsonite oils can be selected for use in this invention by one skilled in the art of drug formulation following the teachings and disclosure herein. In general, the Gilsonite oils desired for use according to this invention will usually have a viscosity in the range of about 5 to about 5000 cps. (at 25° C.), preferably about 5 to about 1000 cps. and more preferably about 5 to about 100 cps. The specific gravity of the Gilsonite oils is usually less than 1.0 and most are in the range of about 0.8 to about 0.95. Of course, the properties of the desired final pharmaceutical formulation for a desired application and function will dictate which oil is selected for each formulation. The Gilsonite oil selected for desired penetration and transport properties can be formulated into a lotion, cream, gel, or adhesive composition, or they can be formulated into liquid or spray compositions containing solvents or permeation enhancers to provide a very low viscosity composition for skin or nail application.

As indicated above, the Gilsonite oils have a range of physical properties, which allow their isolation or purification. Such properties are dictated by the composition of the oils, specifically the individual components and respective percentage composition. The various oils typically comprise one or more of the following individual components or compounds: 6, 10-dimethyl-2-undecanone; 2,6-dimethyl-2,5-heptadien-4-one; 2-butyl-1,1,3-trimethyl cyclohexane; 2,6,10,14-tetramethyl hexadecane; 1,6,7,8,9,9a-hexahydro-4-oxo 4H-pyrido-[1,2-a]pyrimidine-3-carboxylic acid ethyl ester; 2-hydroxy-4,6-dimethylpyrimidine; trans pinane; dihydroedulan; 1-pentyl-2-propyl cyclopentane; 2,3,4-trimethyl 2-cyclopentene-1-one; and, N-formyl-2-phenylpiperidine.

Preferably and conveniently, the Gilsonite oil compositions of this invention which are applied to the skin or nails are formulated with a physiologically acceptable carrier and/or a bioactive agent comprising both the Gilsonite oil and the drug. However, the Gilsonite oil per se may be applied as a pretreatment, prior to the application of the drug formulation which then absorbs the drug formulated for transport through the skin or nails. In such case, a second application of the Gilsonite oil on top of the drug formulation application may be desired for effective drug transport. In such method the pretreatment may be omitted and the drug formulation is applied first, then the Gilsonite oil or oil formulation is applied to the skin or nails. However, such in situ formulation, or multi-step applications are not preferred due to patient inconvenience and complexity. It is preferred that the compositions of this invention be formulated for single-step application. When the formulation includes a carrier, that carrier may comprise any one of conventional topical formulation bases such as those described in Remington: The Science and Practice of Pharmacy, 19th Ed. (Easton, Pa.: Mack Publishing Co., 1995). An oil, lotion, solution, cream, gel, ointment, polymer, adhesive, paste, aerosol, suppository, and nebulized formulation are representative of the topical compositions of this invention. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Particularly preferred formulations herein are ointments, lotions, creams, adhesives and gels.

It is further contemplated that the Gilsonite oils may function as a carrier. An example of such a formulation is a composition comprising Gilsonite Oil and a bioactive agent. The composition is preferably formulated for topical or transdermal application, but other administrative routes such as oral or intraveneous routes, are also possible. Other pharmaceutically acceptable additives as described herein, may optionally be included in a formulation using Gilsonite oil as a carrier.

Further additives for topical formulations are well-known in the art, and may be added to the topical composition, as long as they are pharmaceutically acceptable and not deleterious to the epithelial cells or their function. Further, they should not adversely affect the epithelial penetration efficiency of the Gilsonite oil and should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of transdermal delivery devices as are known in the art.

Other penetration-enhancing compounds are well known in the art, for example, those described in U.S. Pat. Nos. 4,424,210 and 4,316,893, the disclosures of which are incorporated by reference. Preferred penetration-enhancing compounds include 1-dodecylazacycloheptan-2-one (AZONE.RTM.) (Stoughton, Arch. Dermatol., 1982, 118), DMSO, propylene glycol, oleyl alcohol, and methyl pyrrolidone. These additional penetration-enhancing compounds can be used when desired in the composition of this invention in the conventional range of from about 0.1 to about 10% and preferably about 1.0% to about 5.0% by weight of the topical composition.

The formulations of the present invention are topically applied to the body and are adapted for use in specific treatments. For example, to treat pain a formulation is prepared comprising a pain relief agent, Gilsonite oil and a suitable carrier. For another example, to treat disorders associated with the fingernails or toenails (e.g., fungal infections), a formulation is prepared for transporting the appropriate bioactive agent (e.g., antifungal agent) across the nail, or to the skin under the nail, comprising an antifungal agent, Gilsonite oil and a suitable carrier.

There are a number of nail disorders that can be treated with a formulation of the present invention. Such disorders include, without limitation, infections affecting the nails, nail disorders associated with dermatological conditions, acquired nail disorders, periungual and subungual tumors, conditions that require surgical procedures on the nail, and nail disorders associated with general medical conditions. Infections that may affect the nails are onychomychosis (due to dermatophytes, candida or other fungi), paronychia, certain bacterial infections (e.g., due to pseudomonas aeruginosa or other bacteria), viral infections (e.g., periungal or subungal warts), and HIV infection. Nail disorders associated with dermatological conditions include psoriasis, alopecia areata/trachyonychia, eczema, lichen planus, contact dermatitis, 20-nail dystrophy, and pityriasis rubra pilaris. Periungual and subungual tumors embrace superficial basal cell carcinoma and Bowen's disease (i.e., superficial squamous cell carcinoma). Surgical procedures of the nail include nail avulsion and nail biopsy. Nail disorders are frequently associated with a variety of general medical conditions, including, but not limited to, connective tissue diseases, kidney disease and chemotherapy.

A variety of bioactive agents may be used to treat the nail disorders provided above. Onychomycosis is typically treated with an antifungal, urea or a keratolytic agent. Antifungals include allylamines (e.g., naftifine and terbinafine), azoles (e.g., bifonazole, clotrimazole, econazole, fluconazole, itraconazole, ketoconazole, miconazole, oxiconazole, sulconazole, tioconazole, and voriconazole), benzoic acid, benzyl amines (e.g., butenafine), cicloproxolamine, polyenes (e.g., nystatin), thiocarbamates (e.g., tolciclate and tolnaftate) and undecylenic acid; salicylic acid is an example of a keratolytic.

Nail psoriasis may be treated with a corticosteroid, an immunosuppressive, a vitamin D derivative, a retinoid, 5-fluorouracil (i.e., 5-FU), a topical psoralen in combination with UVA light, a keratolytic agent, or urea. Corticosteroids embrace clobetasol proprionate, halobetasol proprionate, betamethasone, diproprionate, diflorasone diacetate, amcinonide, mometasone fuorate, triamcinolone acetonide, fluticasone propionate, flucinolone acetonide, hydrocortisone valerate, flurandrenolide, hydrocortisone butyrate, aclometasone dipropionate desonide, betamethasone valerate, hydrocortisone acetate, and hydrocortisone; cyclosporine, pimecrolimus and tacrolimus are examples of immunosuppressives; vitamin D derivatives include calcipotriol; examples of retinoids are tretinoin and tazarotene; and, keratolytic agents encompass salicylic acid and α-hydroxyacids.

Paronychia and other infections are treated with an antifungal, preferably an azole antifungal, an antibacterial, an antiviral, or sodium sulfacetamide, depending on the nature of the infection. Potential treatments for periungal and subungal warts are imiquimod, a keratolytic agent, bleomycin, or 5-FU. Agents used to treat alopecia areata and lichen planus include corticosteroids, immunosuppressives (e.g., cyclosporine, pimecrolimus, and tacrilumus), retinoids (e.g., tretinoin and tazarotene), and imiquimod. Inflammatory conditions such as eczema and contact dermatitis are usually treated with corticosteroids or topical immunosuppressives. Surgical procedures on the nail typically require the use of anesthetics such as xylocaine, lidocaine and tetracaine. Periungual and subungual tumors may be treated with imiquimod or 5-FU. Bacterial infections, yellow nail syndrome and onychogryphosis are respectively treated with sodium sulfacetamide, a corticosteroid, and TIMS.

Ointments, which are semisolid preparations, are typically based on petrolatum or other petroleum derivatives. As will be appreciated by the ordinarily skilled artisan, the specific ointment base to be used is one that provides for optimum delivery for the bioactive agent chosen for a given formulation, and, preferably, provides for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy, supra, for further information.

Lotions, which are preparations that are to be applied to the skin surface without friction, are typically liquid or semiliquid preparations in which solid particles, including the bioactive agent, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Creams containing a bioactive agent for delivery according to the method of the invention are viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

Gel formulations can also be used in connection with the present invention. As will be appreciated by those working in the field of topical drug formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Useful formulations of the invention also encompass sprays. Sprays generally provide the bioactive agent in an aqueous and/or alcoholic solution which can be misted onto the skin for delivery. Such sprays include those formulated to provide for concentration of the bioactive agent solution at the site of administration following delivery, e.g., the spray solution can be primarily composed of alcohol or other like volatile liquid in which the drug or bioactive agent can be dissolved. Upon delivery to the skin, the carrier evaporates, leaving concentrated bioactive agent at the site of administration.

The pharmaceutical compositions of the present invention are also useful in devices such as patches, strips and containers adapted for topical or transdermal administration. Transdermal drug delivery (TDD) patches are designed to deliver a therapeutically effective amount of drug across a patient's skin. Transdermal patches typically involve a liquid, gel, solid matrix, or pressure-sensitive adhesive carrier into which the drug is incorporated. The earliest TDD systems were reservoir-type devices that used membranes to control the rate of drug release. Today, a drug is more commonly dispersed or dissolved in a pressure-sensitive adhesive (PSA) matrix. Patch formulations and preparation are well known in the art. See for example “Dermatological and Transdermal Formulations” (Drugs and the Pharmaceutical Sciences, Vol 119) by Kenneth A. Walters (Editor), Marcel Dekker and “Transdermal Drug Delivery” (Drugs & the Pharmaceutical Sciences) by Richard H. Guy (Editor), Jonathan Hadgraft (Editor) 2nd Rev& ex edition Marcel Dekker and “Mechanisms of Transdermal Drug Delivery” (Drugs & the Pharmaceutical Sciences, Vol 83) edited by Russell O. Potts and Richard H. Guy (1997).

The pharmaceutical formulations of the invention may be provided as a patch, wherein the drug composition and penetration enhancer are contained within, for example, a laminated structure that serves as a drug delivery device to be affixed to the skin. In such a structure, the pharmaceutical composition is contained within a delivery means, or “reservoir,” which lies beneath an upper backing layer. The laminated structure may contain a single reservoir, or it may contain multiple reservoirs. Patches, i.e., occlusive adhesive devices for transdermal delivery of drugs, are also taught in U.S. Pat. Nos. 4,849,224; 4,983,395; 5,152,997 and 5,302,395. Such devices are generally patches for adhesion to the skin surface and may be in either matrix or reservoir form. Such patches may or may not contain an occlusive backing.

Delivery devices suitable for use in the present invention may be fabricated using conventional techniques, known in the art, for example by casting a fluid admixture of adhesive, bioactive agent, and carrier/vehicle onto the backing layer, followed by lamination of the release liner. Similarly, the adhesive mixture may be cast onto the release liner, followed by lamination of the backing layer. Alternatively, the reservoir may be prepared in the absence of the pharmaceutical formulation or excipient, and then loaded by “soaking” in a drug/vehicle mixture.

The backing layer in the laminates of the patch, which serves as the upper surface of the delivery device, functions as the primary structural element of the laminated structure and may provide the device with flexibility or rigidity, as required by the particular use. The material selected for the backing material should be selected so that it is substantially impermeable to the pharmaceutical formulation to prevent the loss of any components through the upper surface of the device, and to impede dehydration of the composition in the reservoir. The backing layer may be either occlusive or nonocclusive, depending on whether it is desired that the skin become hydrated during drug delivery. The backing is preferably made of a sheet or film of a preferably flexible elastomeric material. Examples of polymers that are suitable for the backing layer include polyethylene, polypropylene, polyesters, and the like.

During storage and prior to use, the patch includes a release liner. Immediately prior to use, this layer is removed from the device to expose the skin-contacting surface of the device, which may be either the reservoir itself or a separate contact adhesive layer, so that the system may be affixed to the skin. The release liner is preferably made of a material that is substantially impermeable to the drug and other components in the formulation, such as polyethylenes and silicones. These liners are well known in the art and are commercially available, for example from 3M Drug Delivery Systems (Minnesota).

Adhesives that are useful in patches are well known in the art (See, for example, S. Venkatraman and R. Gale, “Skin Adhesives and Skin Adhesion, Part I: Transdermal Drug Delivery Systems,” Biomaterials 19, 1119-1136 (1998) and G. Auchter et al., “Acrylic Adhesives,” in Handbook of Pressure Sensitive Adhesive Technology, D. Satas, Ed. (Satas & Associates, Warwick, R.I., 3d ed., 1999), pp. 444-514). Pressure sensitive adhesives are preferably nonirritating, soft and tacky, and have some ability to wet a surface, and to dissolve or complex additives, are stable (ie do not degrade during storage or during the treatment time period) and leave no residue when removed. The most common adhesives used for TDD systems are polymers, and coploymers of acrylates, silicones, and polyisobutylenes. The acrylates can be tailored with appropriate additives to achieve a wide range of performance in regard to various drugs, excipients, and particular product requirements. Some examples of pressure sensitive adhesives include but are not limited to, Duro-Tak® 87-2196, Duro-Tak® 87-2097.

Bioactive agents that can be used with a Gilsonite oil or a mixture of Gilsonite oils include drugs within the broad class normally delivered through body surfaces and membranes, including skin and nail surfaces. In general, this includes therapeutic agents in all of the major areas, including, but not limited to, ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, antipyretics and anti-inflammatory agents, androgens, local and general anesthetics, antiaddictive agents, antiandrogens, antiarrhythmic agents, antiasthmatic agents, anticancer agents, anticholinergic agents, anticholinesterase agents, anticoagulants, antidiabetic agents, antidiarrheal agents, antidiuretic, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinson's agents, antiplatelet agents, antiprogestins, antithyroid agents, antitussives, antiviral agents, atypical antidepressants, azaspirodecanediones, barbituates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, 5-hydroxytryptamine antagonists, drugs for the treatment of hyperlipoproteinemia, hypnotics and sedatives, immunosupressive agents, laxatives, methylxanthines, monoamine oxidase inhibitors, neuromuscular blocking agents, organic nitrates, opioid analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, herb extracts, vitamins and the like.

Representative drugs include, by way of example and not for purposes of limitation, bepridil, diltiazem, felodipine, isradipine, nicardipine, nifedipine, nimodipine, nitredipine, verapamil, dobutamine, isoproterenol, carterolol, labetalol, levobunolol, nadolol, penbutolol, pindolol, propranolol, sotalol, timolol, acebutolol, atenolol, betaxolol, esmolol, metoprolol, albuterol, bitolterol, isoetharine, metaproterenol, pirbuterol, ritodrine, terbutaline, alclometasone, aldosterone, amcinonide, beclomethasone dipropionate, betamethasone, clobetasol, clocortolone, cortisol, cortisone, corticosterone, desonide, desoximetasone, 11-desoxycorticosterone, 11-desoxycortisol, dexamethasone, diflorasone, fludrocortisone, flunisolide, fluocinolone, fluocinonide, fluorometholone, flurandrenolide, halcinonide, hydrocortisone, medrysone, 6.alpha.-methylprednisolone, mometasone, paramethasone, prednisolone, prednisone, tetrahydrocortisol, triamcinolone, benoxinate, benzocaine, bupivacaine, chloroprocaine, cocaine, dibucaine, dyclonine, etidocaine, lidocaine, mepivacaine, pramoxine, prilocaine, procaine, proparacaine, tetracaine, alfentanil, choroform, clonidine, cyclopropane, desflurane, diethyl ether, droperidol, enflurane, etomidate, fentanyl, halothane, isoflurane, ketamine hydrochloride, meperidine, methohexital, methoxyflurane, morphine, propofol, sevoflurane, sufentanil, thiamylal, thiopental, acetaminophen, allopurinol, apazone, aspirin, auranofin, aurothioglucose, colchicine, diclofenac, diflunisal, etodolac, fenoprofen, flurbiprofen, gold sodium thiomalate, ibuprofen, indomethacin, insulin, ketoprofen, meclofenamate, mefenamic acid, meselamine, methyl salicylate, nabumetone, naproxen, nicotine, oxyphenbutazone, phenacetin, phenylbutazone, piroxicam, salicylamide, salicylate, salicylic acid, salsalate, sulfasalazine, sulindac, tolmetin, acetophenazine, chlorpromazine, fluphenazine, mesoridazine, perphenazine, thioridazine, trifluorperazine, triflupromazine, disopyramide, encainide, flecainide, indecainide, mexiletine, moricizine, phenytoin, procainamide, propafenone, quinidine, tocainide, cisapride, domperidone, dronabinol, haloperidol, metoclopramide, nabilone, prochlorperazine, promethazine, thiethylperazine, trimethobenzamide, buprenorphine, butorphanol, codeine, dezocine, diphenoxylate, drocode, hydrocodone, hydromorphone, levallorphan, levorphanol, loperamide, meptazinol, methadone, nalbuphine, nalmefene, nalorphine, naloxone, naltrexone, oxybutynin, oxycodone, oxymorphone, pentazocine, propoxyphene, isosorbide dinitrate, nitroglycerin, theophylline, phenylephrine, ephidrine, pilocarpine, furosemide, tetracycline, chlorpheniramine, ketorolac, bromocriptine, guanabenz, prazosin, doxazosin, and flufenamic acid.

Other representative drugs include benzodiazepines, such as aiprazolam, brotizolam, chlordiazepoxide, clobazam, clonazepam, clorazepate, demoxepam, diazepam, flumazenil, flurazepam, halazepam, lorazepam, midazolam, nitrazepam, nordazepam, oxazepam, prazepam, cluazepam, temazepam, triazolam, and the like; an antimuscarinic agent such as anisotropine, atropine, clidinium, cyclopentolate, dicyclomine, flavoxate, glycopyrrolate, hexocyclium, homatropine, ipratropium, isopropamide, mepenzolate, methantheline, oxyphencyclimine, pirenzepine, propantheline, scopolamine, telenzepine, tridihexethyl, tropicamide, and the like; an estrogen such as chlorotrianisene, siethylstilbestrol, methyl estradiol, estrone, estrone sodium sulfate, estropipate, mestranol, quinestrol, sodium equilin sulfate, 17.beta.-estradiol (or estradiol), semi-synthetic estrogen derivatives such as the esters cf natural estrogen, such as estradiol-17.beta.-enanthate, estradiol-17.beta.-valerate, estradiol-3-benzoate, estradiol-17.beta.-undecenoate, estradiol 16,17-hemisuccinate or estradiol-17.beta.-cypionate, and the 17-alkylated estrogens, such as ethinyl estradiol, ethinyl estradiol-3-isopropylsulphonate, and the like; an androgen such as danazol, fluoxymesterone, methandrostenolone, methyltestosterone, nandrolone decanoate, nandrolone phenpropionate, oxandrolone, oxymetholone, stanozolol, testolactone, testosterone, testosterone cypionate, testosterone enanthate, testosterone propionate, and the like; or a progestin such as ethynodiol diacetate, gestodene, hydroxyprogesterone caproate, levonorgestrel, medroxyprogesterone acetate, megestrol acetate, norethindrone, norethindrone acetate, norethynodrel, norgestrel, progesterone; immunosuppressives, such as cyclosporins and the like.

The amount of the drug that is present in the formulation, and that is required to achieve a therapeutic effect, depends on many factors, such as the minimum necessary dosage of the particular drug and will most likely be determined by a physician, in the light of the relevant circumstances, including the condition to be treated, the chosen route of administration, the actual compound administered, the age, weight, and response of the individual patient, the severity of the patient's symptoms, and the like.

The appropriate dosage level of the physiologically active substance, without the use of the penetration enhancing formulations of the present invention, are known to one skilled in the art. These conventional dosage levels correspond to the upper range of dosage levels for compositions, including a physiologically active substance and traditional penetration enhancer. However, because the delivery of the active substance is enhanced by the Gilsonite Oil Extracts, desired dosage levels can be achieved with formulations containing significantly lower drug concentration than conventional formulations. In general, the active substance will be present in the composition in an amount from about 0.0001% to about 60%, more preferably about 0.001% to about 20% by weight of the total composition depending upon the particular substance employed. However, generally the amount will range from about 0.01 to about 25% by weight of the total composition, with levels of from about 0.05 to about 10% being preferred.

Topical treatment regimens according to the practice of this invention comprise applying the composition directly to the skin or mucosa, i.e., at the application site, from one to several times daily.

Formulations of the present invention can be used to treat, ameliorate or prevent diseases and conditions, or the symptoms associated with such diseases and conditions, such as pain, psoriasis, dry skin, Rosaceae, irritation, sun burn, fungal infections, bacterial infections, baldness, hormone imbalance, and the like. In addition the present invention can be used in contraception formulations.

The major function of epithelia, including the stratum corneum of epidermis and of keratinizing mucous membranes, is to prevent the excessive loss of bodily fluids. If the epithelial barrier function is disrupted or perturbed, it stimulates a variety of metabolic changes in the epidermis and mucous membranes leading to repair of the barrier defect. While the barrier is beneficial for protection against damage from ultraviolet radiation, desiccation, chemical, frictional, and blunt trauma, it also impedes the percutaneous and transmucosal penetration of topically applied medicaments of potential benefit to the host. The inability of physiologically active agents to penetrate the epithelium significantly limits their effective use for treating disease conditions and disorders not only of the skin and mucosae, but also of a systemic nature. The compositions of this invention enhance the delivery of both water soluble and lipid soluble agents.

As discussed above, a variety of pharmaceutical formulations have been developed with varying degrees of success, that allow for transdermal treatment. It is known to those skilled in the art that, among other factors, there is a size dependence in the rate at which molecules are transported across the epithelial barrier. The flux of larger molecules across the barrier is typically lower than for small molecules. Examples 3 through 5 below demonstrate that Gilsonite oils are effective penetration enhancers for small, medium and larger molecules. Caffeine, hydrocortisone, and a series of PEG molecules, with molecular weight from 502 to 766, were chosen as model compounds for small, medium and large molecules, respectively. Results obtained with a series of PEG molecules show decreased permeation with increased molecular weight, or size, in the presence and absence of Gilsonite oils. Pretreatment with Gilsonite oils resulted in substantially greater flux for all PEG molecules studies.

Pharmaceutical compositions comprising a Gilsonite oil allows for enhanced penetration relative to treatment with the drug formulation alone or in combination with a state of the art pretreatment formulation, such as DMSO as a pretreatment before application of the Gilsonite oil composition, as a post treatment after application of the Gilsonite oil composition, or included in and as part of the Gilsonite oil composition. Pharmaceutical formulations comprising a Gilsonite oil and a bioactive agent allow for enhanced penetration of the bioactive agent compared to a drug formulation with no Gilsonite oil or with a drug formulation comprising a state of the art penetration enhancer, such as DMSO. The penetration enhancing properties of a Gilsonite oil formulation are effective for bioactive agents, whether they are small molecules, medium molecules or large molecules.

Initial screening of the Gilsonite formulations is done using conventional Transepidermal Water Loss (TEWL) tests using mice. For example, each oil sample can be applied topically to the mice twice daily, for four days. For samples that are liquids at room temperature a sample of about 40 to about 80 μL of the oil is applied to the skin surface. For samples that are solid at room temperature a sample of about 40 to about 60 mg of the oil is applied to the skin surface. The TEWL and skin hydration are measured using techniques well known in the art using a Meeco device (Warrington, Pa.) and a Corneometer (model CM-820, Courage & Khazak, Germany), respectively.

Subsequent evaluation of Gilsonite oil formulations is done using skin penetration studies. Skin penetration studies can performed using flow-through diffusion cells (Laboratory Glass Apparatus, Berkeley, Calif.). The diffusion cells are maintained at a constant temperature using a circulating water bath. The skin is mounted on the diffusion cell with the epidermal side toward the receiver chamber. An isotonic solution is pumped through the receiver compartment at a flow rate of 3-4 mL/h using a peristaltic pump (Ismatec, Glattbrug-Zürich, Switzerland). If the outer skin surface is pretreated residual pretreatment solution is removed and the skin surface is washed

A test solution containing a suitable amount of radioactively labeled test compound can be applied to the skin surface, and the upper donor cell is then sealed with parafilm. In a positive control experiment, skin can be pretreated with dimethyl sulfoxide (DMSO) for 2 h. The receiver fluid is stirred and samples are collected at designated time intervals over a period of about 12 hours with a Retriever IV fraction collector (ISCO, Lincoln, Neb.). Each fraction is mixed with Ready Safe®, and analyzed in a Beckman LS 5000TA liquid scintillation counter. Flux through the skin is calculated from the slope of the linear part of the cumulative amount of drug penetrated versus time curve.

While the present invention has been described with respect to preferred embodiments thereof, it will be understood that various changes and modifications will be apparent to those skilled in the art, and that it is intended that the invention encompass such changes and modifications as failing within the scope of the appended claims. The following non-limiting examples are provided to further illustrate the present invention.

### EXAMPLES

In the examples below, the following abbreviations have the following meanings. If an abbreviation is not defined, it has its generally accepted meaning.
cps=centipoise
EtOH=ethanol
h=hours
HPLC=high pressure liquid chromatography
M=molar
mg=milligram
min.=minutes
mL=milliliter
mm=millimeter
mM=millimolar
mmol=millimole
N=normal
NS=normal saline
psi=pounds per square inch
PEG=polyethylene glycol
μm micrometer
μM=micromolar
μL=microliter
% mol=mol percent
TEWL=Transepidermal Water Loss

In the following examples and procedures, the conventional compounds and reagents used in the procedures are commercially available from well-known sources. The Gilsonite oils identified and used in the Examples were obtained from American Gilsonite Company, San Francisco, Calif..

### Example 1

### Determination of the Effect of Gilsonite Oil on Transepidermal Water Loss (TEWL) and Skin Hydration Levels.

One of the primary functions of skin is to retain water within the body. Hence, transepidermal water loss (TEWL) and skin hydration values are useful tools for assessing barrier function. In the experiments described here skin hydration and TEWL were measured in hairless mice following repeated application of the oils tested.

Four mice were tested for each oil sample, which was applied topically to the mice twice daily, for four days. For samples that were liquids at room temperature 60 μL of the oil was applied to the skin surface. For samples that were solid at room temperature, about 60 mg of the oil was applied to the skin surface. The TEWL was measured with a Meeco device (Warrington, PA) and skin hydration was measured with a Corneometer (model CM-820, Courage & Khazak, Germany). The results show a consistent drop in hydration (Table 1) and increase in TEWL (Table 2) with repeated oil treatment for all oils studied. The decline in hydration and increase in TEWL is statistically significant (T-test, p>0.95) when the last day of measurement is compared to the pre-treatment value obtained on day 0.

TABLE 1
Hydration values following treatment of hairless mouse skin
with various oils. The oil sample was applied twice daily for
the first four days. The values presented are the average
and standard error of the mean (SEM) for all determinations.

| Day | | Oil A | Oil B | Oil C | Oil Q | Oil X | Oil Y | Oil 1 | Oil 2 |
|---|---|---|---|---|---|---|---|---|---|
| 0 | Avg | 58 | 58 | 59.1 | 56.3 | 62.5 | 62.5 | 53.8 | 53.8 |
| | SEM | 1.2 | 1.2 | 1.3 | 1.9 | 3.5 | 3.5 | 0.59 | 0.59 |
| 1 | Avg | — | — | — | — | — | — | 43.4 | 48.8 |
| | SEM | — | — | — | — | — | — | 1.1 | 0.75 |
| 2 | Avg | 52.8 | 36.4 | — | — | 58.5 | 54 | 28.3 | 41.6 |
| | SEM | 1.0 | 2.1 | — | — | 2.6 | 0.4 | 1.7 | 1.12 |
| 3 | Avg | 38 | 23.3 | 46.8 | 25.8 | 38.5 | 17.8 | 30.3 | 38.0 |
| | SEM | 2.5 | 2.1 | 2.1 | 1.4 | 1.3 | 3.0 | 2.6 | 2.0 |
| 4 | Avg | 20 | 18.4 | — | — | 18.5 | 12.3 | 14.6 | 22.8 |
| | SEM | 0.8 | 2.0 | — | — | 2.9 | 1.4 | 1.2 | 1.3 |
| 5 | Avg | — | — | 25.9 | — | — | — | — | — |
| | SEM | — | — | 2.1 | — | — | — | — | — |
| 7 | Avg | — | — | 18.8 | — | — | — | — | — |
| | SEM | — | — | 1.4 | — | — | — | — | — |

TABLE 2
TEWL values following treatment of hairless mouse skin with various oils. The oil
sample was applied twice daily for the first four days. The values presented are
the average and standard error of the mean (SEM) for all determinations.

| Day | | Oil A | Oil B | Oil C | Oil Q | Oil X | Oil Y | Oil 1 | Oil 2 |
|---|---|---|---|---|---|---|---|---|---|
| 0 | Avg | 17.3 | 17.3 | 17.4 | 19.0 | 17.3 | 17.3 | 17.3 | 17.3 |
| | SEM | 0.53 | 0.53 | 0.7 | 0.7 | 0.53 | 0.53 | 0.53 | 0.53 |
| 1 | Avg | — | — | — | — | — | — | 56.3 | 37.0 |
| | SEM | — | — | — | — | — | — | 39.2 | 3.8 |
| 2 | Avg | 27.5 | 43.0 | — | — | 17.3 | 15.0 | 303 | 184 |
| | SEM | 2.5 | 9.3 | — | — | 2.6 | 2.1 | 70.3 | 9.3 |
| 3 | Avg | 92.0 | 441 | 243 | 940 | 38.5 | 67.5 | 648 | 325 |
| | SEM | 31.5 | 108 | 43.6 | 35.1 | 1.3 | 98.4 | 61.0 | 19.2 |
| 4 | Avg | 345 | 616 | — | — | 475 | 473 | 588 | 704 |
| | SEM | 5.6 | 58.0 | — | — | 23.4 | 34.0 | 60.1 | 60.9 |
| 5 | Avg | — | — | 353 | — | — | — | — | — |
| | SEM | — | — | 42.6 | — | — | — | — | — |
| 7 | Avg | — | — | 438 | — | — | — | — | — |
| | SEM | — | — | 40.8 | — | — | — | — | — |

These results show that all topically applied oils tested increase TEWL and lower skin hydration consistent with increased water transport through the treated skin.

### Example 2

### Effect of the Gilsonite Oil on the Skin Penetration of PEG 600.

Abdominal porcine skin was obtained from a local abattoir. Immediately after obtaining the skin, the hair was clipped and the skin was sectioned with a dermatome to 500 μm thickness. Sheets of dermatomed skin were sandwiched between tissue paper soaked with normal saline (NS) and stored at −70° C. before use.

Skin penetration studies were performed using flow-through diffusion cells (Laboratory Glass Apparatus, Berkeley, Calif.) with a diffusion area of 1 cm² and a receiver volume of 3.6 mL. The diffusion cells were maintained at a constant temperature of 37° C. using a circulating water bath.

Oil samples for diffusion measurements were prepared by mixing an equal volume of the oil with ethanol (EtOH). After equilibration the supernatant was used for skin pretreatment. The skin was mounted on a diffusion cell with the epidermal side toward the receiver chamber. An isotonic solution (pH 7.4, phosphate buffered saline—PBS) was pumped through the receiver compartment at a flow rate of 3-4 mL/h using a peristaltic pump (Ismatec, Glattbrug-Zürich, Switzerland). The outer skin surface was pretreated for 24 h with 500 μL of the oil/EtOH solution or a control solution of H₂O/EtOH (1:1 by volume). At the end of 24 h, residual solution was removed and the skin surface washed twice with 200 μL of EtOH and once with 200 μL of NS.

After skin pretreatment and washing, the outlet of each diffusion cell was connected to its own inlet to close the receiver compartment. A solution (500 μL) of 0.25 M PEG 600 in NS was applied to the skin surface and the upper donor cell was sealed with parafilm. The PEG 600 is a mixture of polyethylene glycol oligomers. The receptor fluid was stirred at 700 rpm with a small Teflon-coated magnet. At the end of 12 h time period, the entire receiver fluid was emptied and collected in a glass tube. In a positive control experiment, skin was pretreated with dimethyl sulfoxide (DMSO) for 2 h.

Separation and quantitation of individual oligomers in PEG 600 samples was accomplished using a high performance liquid chromatographic (HPLC). The HPLC system consisted of a Model 510 pump (Waters Co., Milford, Mass., U.S.A.), and a 717 plus autosampler (Waters) on a C8 reverse column (Microsorb-MV_{C}, 5 μm, 4.6×250 mm, Ranin Instrument, Woburn, Mass., U.S.A.). A mobile phase of methanol-water (40:60 by volume) was delivered at a flow rate of 1 mL/min. A Series 200 refractive index detector (Perkin Elmer Co., Norwalk, Conn., U.S.A.) was used to detect the PEG oligomers as they eluted from the column. The refractive index detector was maintained at a constant temperature at 35° C.

At the end of 12-h time period, the entire receiver fluid was emptied and collected in 15 mL Pyrex tube, for subsequent lyophilization and extraction. Following lyophilization the PEG was extracted by adding 10 mL of chloroform to the sample followed by vortexing for 1 min at high speed. The solvent was then allowed to remain in contact with the sample for at least 30 minutes. The sample was centrifuged at 3000 rpm for 10 min, and the supernatant was transferred to clean tube. Chloroform extraction was repeated a second time, and the combined supernatant was evaporated to dryness at 50° C. under a stream of N₂ gas. The residue was then dissolved in 1 mL of HPLC mobile phase. This sample was injected in the HPLC for analysis.

Assay standards were prepared by lyophilizing 1 mL of PEG 600 in PBS, ranging in concentration from 0.05 to 10 mg/mL, followed by extraction as described above. After injection in the HPLC column the refractive index peak area of each oligomer was plotted against the polymer concentration of the standards. Regression equations relating peak area to concentration were calculated for each oligomer. The peak area of each oligomer in the unknown samples was calculated and converted to the relative amount of PEG present using the regression equations. The largest peak from the chromatogram of PEG 600 standard was assigned to the oligomer with MW closest to the average value, i.e., oligomer with MW of 590 Da. A total of 14 oligomers were detected in PEG 600 samples. However, because of limited quantities and associated low signal, only data for oligomers with MW between 502 and 766 Da were determined. Extraction recovery ranged from approximately 60% to 103%.

As seen in the table below, Oils B & Y produced a statistically significant increase in PEG flux relative to the control.

Skin Penetration of PEG 600
The Values are presented as the
mean ± one standard deviation.

| | Total Penetration | |
|---|---|---|
| Composition | in 12 h (% of applied) | Ratio |
| Control | 0.110 ± 0.041 | 1.00 ± 0.37 |
| DMSO | 0.882 ± 0.742 | 8.25 ± 6.73 |
| Gilsonite | 2.768 ± 2.740 | 26.17 ± 24.85 |
| Oil B | | |
| Oil X | 0.346 ± 0.300 | 3.09 ± 2.72 |
| Oil Y | 3.390 ± 3.610 | 31.87 ± 32.74 |
| Oil Q | 1.563 ± 1.204 | 14.40 ± 10.92 |

### Example 3

### The Effect of Gilsonite Oils on the Skin Penetration of Hydrocortisone.

The experimental setup and skin pretreatment procedures were the same as described in Example 2 above. At the end of the 24 h pretreatment process, the residual solution was removed and the skin surface washed twice with 200 μL of EtOH and once with 200 μL of NS. A hydrocortisone (0.1 mg/mL in NS) solution (500 μL) containing a suitable amount of radioactively labeled compound (³H-hydrocortisone) was applied to the skin surface, and the upper donor cell was sealed with parafilm. In a positive control experiment, skin was pretreated with dimethyl sulfoxide (DMSO) for 2 h. The receiver fluid was stirred at 700 rpm with a small Teflon-coated magnet. Samples were collected at 1-h interval for 12 h with a Retriever IV fraction collector (ISCO, Lincoln, Neb.). Each fraction was mixed with 10 mL Ready Safe®, and analyzed in a Beckman LS 5000TA liquid scintillation counter. Flux through the skin was calculated from the slope of the linear part of the cumulative amount of drug penetrated versus time curve.

As seen in the table below, a statistically significant increase in flux relative to the Control, decrease in lag time and increase in 12-hour permeation was seen with Oils 1, A, B, C, X, Y and Q.

Skin Penetration of Hydrocortisone Composition
The Values are presented as the mean ± one standard deviation. N = number of replicates.

| | DMSO | Control | Oil-1 | Oil-2 | Oil-A | Oil-B | Oil-C | Oil-X | Oil-Y | Oil-Q |
|---|---|---|---|---|---|---|---|---|---|---|
| Flux (μg/cm²/h) | 0.0595 | 0.0640 | 0.1077 | 0.0423 | 0.1862 | 1.1163 | 0.5178 | 0.6172 | 0.8547 | 0.8227 |
| SD | | 0.0353 | 0.0318 | 0.0337 | 0.1419 | 0.1012 | 0.1656 | 0.0922 | 0.1746 | 0.0611 |
| Lag time (hr) | 3.4017 | 3.6505 | 1.5063 | 3.1672 | 1.6475 | 0.6935 | 1.0449 | 1.1144 | 1.2020 | 1.0417 |
| SD | | 0.8566 | 0.2256 | 0.2986 | 0.4360 | 0.3929 | 0.3365 | 0.0982 | 0.4974 | 0.1593 |
| 12 h penetration | 0.5318 | 0.5640 | 1.1298 | 0.3881 | 1.9707 | 12.2450 | 5.6183 | 6.7937 | 9.1363 | 8.8787 |
| (μg) | | | | | | | | | | |
| SD | | 0.3460 | 0.3767 | 0.3166 | 1.5109 | 1.0872 | 1.8550 | 1.0424 | 1.9076 | 0.5723 |
| N | 1 | 11 | 4 | 4 | 5 | 6 | 4 | 3 | 4 | 4 |

### Example 4

### The Effect of Gilsonite Oils on the Skin Penetration of Caffeine.

The experimental setup and skin pretreatment procedures were the same as described in Example 3 above. At the end of the 24 h pretreatment process, the residual solution was removed and the skin surface washed twice with 200 μL of EtOH and once with 200 μL of NS. A caffeine (5.3 mg/mL in NS) solution (500 μL) containing a suitable amount of radioactively labeled compound (¹⁴C-caffeine) was applied to the skin surface, and the upper donor cell was sealed with parafilm. In a positive control experiment, skin was pretreated with dimethyl sulfoxide (DMSO) for 2 h. The receiver fluid was stirred at 700 rpm with a small Teflon-coated magnet. Samples were collected at 1-h interval for 12 h with a Retriever IV fraction collector (ISCO, Lincoln, Neb.). Each fraction was mixed with 10 mL Ready Safe®, and analyzed in a Beckman LS 5000TA liquid scintillation counter. Flux through the skin was calculated from the slope of the linear part of the cumulative amount of drug penetrated versus time curve.

As seen in the table below, a statistically significant increase in flux relative to the control was seen with all but Oil-2. A statistically significant decrease in lag time and increase in 12-hour permeation was seen with all oils.

Skin Penetration Caffeine Compositions
The Values are presented at the mean ± one standard deviation. N = number of replicates.

| | DMSO | Control | Oil-1 | Oil-2 | Oil-A | Oil-B | Oil-C | Oil-X | Oil-Y | Oil-Q |
|---|---|---|---|---|---|---|---|---|---|---|
| Flux (μg/cm²/h) | 10.54 | 3.64 | 41.49 | 2.39 | 17.66 | 20.19 | 93.52 | 43.31 | 41.00 | 84.61 |
| SD | | 2.03 | 23.47 | 0.77 | 9.43 | 8.46 | 13.93 | 17.43 | 19.57 | 10.57 |
| Lag time (hr) | 2.21 | 2.70 | 0.81 | 1.68 | 0.98 | 0.98 | 0.54 | 0.50 | 0.79 | 0.92 |
| SD | | 0.72 | 0.24 | 0.36 | 0.22 | 0.25 | 0.16 | 0.37 | 0.23 | 0.18 |
| 12 h pentration | 104.85 | 36.22 | 460.54 | 25.53 | 197.02 | 224.15 | 1041.50 | 489.21 | 456.76 | 924.81 |
| (μg) | | | | | | | | | | |
| SD | | 22.95 | 262.68 | 8.64 | 105.32 | 95.81 | 157.30 | 185.01 | 221.53 | 112.89 |
| N | 1 | 13 | 4 | 6 | 8 | 5 | 5 | 5 | 6 | 4 |

The disclosure presented herein is intended to be an enabling and illustrative disclosure for the inventive concepts of this invention and is not intended to be limiting in the scope of the invention, which is set forth in the following claims.

What is claimed is:

1. A composition for topical or transdermal administration comprising:
(i) a bioactive agent, wherein the bioactive agent is selected to treat a nail disorder; and
(ii) Gilsonite oil.

2. The composition of claim 1, further comprising a pharmaceutically acceptable carrier.

3. The composition of claim 2, further comprising a pharmaceutically acceptable additive selected from the group consisting of excipients, preservatives, antioxidants, fragrances, emulsifiers, dyes, adhesives, polymers and additional penetration enhancers.

4. The composition of claim 1, wherein the bioactive agent is present in about 0.0001% to about 50% by weight.

5. The composition of claim 1, wherein the Gilsonite oil is present in about 0.0001% to about 50% by weight.

6. A method for enhancing the penetration of a bioactive agent through the nail of a human or warm-blooded animal which comprises applying to a body surface:
(a) a bioactive agent; and
(b) a penetration-enhancing system comprising,
(i) Gilsonite oil; and
(ii) a pharmaceutically acceptable carrier.

7. The method for enhancing the penetration of claim 6, wherein the pharmaceutically acceptable carrier is selected from the group consisting of oils, lotions, gels, creams, adhesives and ointments.

8. The method for enhancing the penetration of claim 6, wherein the pharmaceutically acceptable carrier is suitable for use with transdermal patches.

9. The method for enhancing the penetration of claim 7, further comprising a pharmaceutically acceptable additive selected from the group consisting of excipients, preservatives, antioxidants, fragrances, emulsifiers, dyes, adhesives, polymers and additional penetration enhancers.

10. The method for enhancing the penetration claim 7, wherein the bioactive agent is present in about 0.0001% to about 50% by weight.

11. The method for enhancing the penetration of claim 7, wherein the Gilsonite oil is present in about 0.0001% to about 50% by weight.

12. A method for delivering a bioactive agent transdermally by administering to the nail of an animal a composition comprising
(i) a bioactive agent wherein the bioactive agent is selected to treat a nail disorder; and
(ii) Gilsonite oil.

13. The method for delivering a bioactive agent transdermally of claim 12, further comprising a pharmaceutically acceptable carrier selected from the group consisting of oils, lotions, gels, creams, and ointments.

14. The method for delivering a bioactive agent transdermally of claim 12, further comprising a pharmaceutically acceptable additive selected from the group consisting of excipients, preservatives, antioxidants, fragrances, emulsifiers, dyes, adhesives, polymers and additional penetration enhancers.

15. The method for delivering a bioactive agent transdermally of claim 12, wherein the bioactive agent is present in about 0.0001% to about 50% by weight.

16. The method for delivering a bioactive agent transdermally of claim 12, wherein the Gilsonite oil is present in about 0.0001% to about 50% by weight.

17. A method for making a composition for transdermal delivery of a bioactive agent comprising mixing the bioactive agent and a Gilsonite oil in proportions effective to provide a composition for the desired use, wherein the bioactive agent is selected to treat a nail disorder.

18. The method for making a composition for transdermal delivery of claim 17, further comprising mixing in a pharmaceutically acceptable carrier selected from the group consisting of oils, lotions, gels, creams, adhesives and ointments.

19. The method of claim 17, further comprising mixing in at least one pharmaceutically acceptable penetration-enhancing additives, excipients, preservatives, antioxidants, fragrances, emulsifiers, or dyes.
